(19) United States
(12) Patent Application Publication
(10) Pub. No.: US 2011/0074585 A1
(43) Pub. Date: Mar. 31, 2011

(54) PATIENT TRACKING SYSTEM

(75) Inventors: J. Douglas Harmon, Lincolnton, GA (US); Alan B. Whitehouse, Augusta, GA (US); W. Andrew Wells, Augusta, GA (US); Keith A. Lynn, Johnson, SC (US); Stephen W. Smith, Martinez, GA (US)

(73) Assignee: AUGUSTA E.N.T., P.C., Evans, GA (US)

(21) Appl. No.: 12/883,209

(22) Filed: Sep. 16, 2010

Related U.S. Application Data
(60) Provisional application No. 61/246,263, filed on Sep. 28, 2009.

(51) Int. Cl. G08B 23/00; G06F 3/048; G06F 15/16

(52) U.S. Cl. 340/573.1; 715/738

(57) ABSTRACT

Provided are a method and system for tracking a patient at a medical facility. Tracking the patient includes, using a computer comprising an input peripheral manipulated by a user, receiving a first location of the patient within the medical facility identified by the user via the input peripheral. The first location is displayed on a display device operatively connected to the computer, the first location being identified by a graphical marker representing the first location where the patient can be found. In response to a user interaction with the input peripheral, a second location of the patient is received, indicating movement of the patient from the first location to the second, different location. In response to receiving the second location, the graphical marker on the display device operatively connected to the computer is moved to identify the second location where the patient can be found after being moved.

This application claims the benefit of U.S. Provisional Application No. 61/246,263, filed Sep. 28, 2009, which is incorporated in its entirety herein by reference.

### BACKGROUND OF THE INVENTION

1. Field of the Invention

This application relates generally to a method and apparatus for tracking patients at a medical treatment facility and, more specifically, to a computerized method and apparatus for presenting a graphical user interface to graphically depict a patient's location within the medical treatment facility.

2. Description of Related Art

The number of patients that visit medical offices seeking medical treatment continues to grow each year. A large number of patients can make it difficult for physicians and support staff to track the location of each patient at the medical office to ensure timely treatment and efficient use of the physicians' time.

For example, a medical office with ten practicing physicians can average about 4,500 patient visits per year. If departmentalized into two or more departments catering to the medical needs of patients, that same medical office with ten physicians may serve more than an additional 3,000 patients annually. For example, the medical office may include two allergy departments, each with three Allergists, and an audiology department with four Audiologists that each average about 20 visits per day. Patients may even visit a medical professional in more than one department during any given visit to the medical office. Thus, the large number of patients, as well as the layout of the medical office itself can present logistical challenges in tracking the location and movement of patients during the normal course of a day. Time spent locating patients amounts to a waste of the medical professionals' time, and can also result in lengthy delays in treatment, which can be a source of frustration on the part of the patients.

Historically tracking patients has been an intense process that employs visual queues such as lights over doors, physical files hanging on doors or sticky notes left on a desk. Each of these systems is labor intensive, not very well coordinated, and prone to errors.

### BRIEF SUMMARY

Accordingly, there is a need in the art for a method and system for tracking patients at a medical office. Such a method and system can include a computer system executing computer-executable logic stored in an electronic format on a computer readable medium to provide a visual presentation of patient locations within a medical office, and can optionally graphically present patient locations on a graphical layout representing a plurality of rooms associated with a physician.

According to one aspect, the subject application involves a method of tracking a patient at a medical facility. The method includes receiving, from a computer terminal, a first location of the patient within the medical facility, and storing the first location occupied by the patient in a computer-readable memory that is accessible to the first computer terminal over a communication network. Content indicating that the patient is occupying the first location is transmitted over the communication network to a remotely-located terminal comprising a display device that is operable to display a graphic indicating that the patient is occupying the first location. A second location of the patient is received, indicating movement of the patient from the first location to the second location, which is different than the first location. In response to receiving the second location, storing the second location occupied by the patient in the computer-readable memory. Content indicating that the patient is occupying the second location is then transmitted over the communication network to the remotely-located terminal for updating the graphic displayed by the remotely-located terminal to reflect movement of the patient from the first location to the second location.

According to another aspect, the subject application involves a method of tracking a patient at a medical facility. The method according to the present aspect, includes, using a computer comprising an input peripheral manipulated by a user, receiving a first location of the patient within the medical facility identified by the user via the input peripheral. The first location is displayed on a display device operatively connected to the computer, the first location being identified by a graphical marker representing the first location where the patient can be found. The method also includes, in response to user interaction with the input peripheral, receiving a second location of the patient indicating movement of the patient from the first location to the second location, which is different than the first location. In response to receiving the second location, the graphical marker on the display device operatively connected to the computer is moved to identify the second location where the patient can be found after being moved.

According to another aspect, the subject application involves a system for tracking a location of a patient. The system includes a computer terminal operatively connected to a communication network, and a remotely-located computer terminal that is also operatively connected to the communication network. The computer terminal and the remotely-located computer terminal each comprise a display device and are disposed at different locations within a medical facility. A computer-readable memory is accessible by at least one of the computer terminal and the remotely-located computer terminal over the communication network. The computer-readable memory is operable to store information indicative of the location of the patient within the medical facility, and each of the computer terminal and the remotely-located computer terminal comprise a memory storing computer-executable instructions to be executed for performing a method. The method includes receiving from the computer-readable memory information indicative of the location of the patient within the medical facility. In response to receiving the information, a graphical representation of a plurality of different examination locations at the medical facility is presented, each of the plurality of different examination locations being represented by a room icon. An occupied room icon representing one of the plurality of examination locations occupied by the patient includes an appearance that is visually distinguishable from an unoccupied room icon representing another of the plurality of examination locations not occupied by the patient. A movement of the patient from a first examination location to a second examination location is entered. Information indicative of said movement is then transmitted over the communication network to update the graphical representation of the plurality of different examination locations displayed by the remotely-located computer terminal.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF THE DRAWINGS

The invention may take physical form in certain parts and arrangement of parts, embodiments of which will be described in detail in this specification and illustrated in the accompanying drawings which form a part hereof and wherein:

FIG. 1 schematically depicts an illustrative embodiment of a computer for implementing a method of tracking patients in a medical office;

FIG. 2 shows an illustrative embodiment of an administrative window generated by a computer system and presented to a technician to enable selection of a basis for tracking patients in a medical office;

FIG. 3 shows an illustrative embodiment of a layout window generated by a computer system and presented to a technician to enable the establishment of a reference point;

FIG. 4 shows an illustrative embodiment of a room identification window generated by a computer system and presented to a technician to enable arrangement of examination rooms relative to a reference point; and

FIG. 5 shows an illustrative embodiment of a monitoring window generated by a computer system and presented to a technician to enable graphical determination of a patient's location.

### DETAILED DESCRIPTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

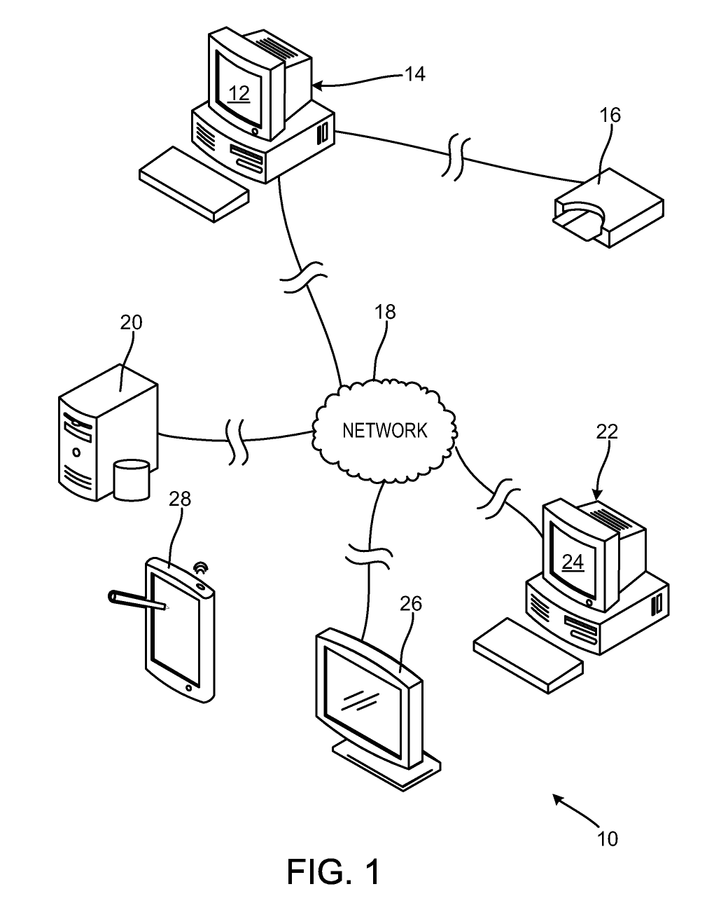

Provided is a computer system 10 and method for generating a virtual representation of a medical office to provide physicians, nurses, physician assistants, support staff and other medical office personnel (generally referred to herein as “technicians”) with a graphical user interface (“GUI”) indicating a location of one or more patients within the medical office. The virtual representation described herein includes an image, such as a photograph of the patient for example, displayed by a display device 12 provided to a computer terminal 14 such as that operated by a receptionist or other administrative personnel as shown in FIG. 1 for example. The computer terminal 14 can optionally be located at a reception/waiting location within the medical office where patients arrive and await their turn to be treated. Patients can optionally be checked in by scanning an ID card using an input peripheral such as a scanner 16 that can read a computer-readable code such as a barcode, RFID tag, magnetic strip, etc. . . . The scanner can optionally be locally connected to the computer terminal 14 as shown, but alternate embodiments include the scanner 16 being operatively connected to the computer terminal 14 via a communications network 18 such as a local area network (“LAN”), wide area network (“WAN”) such as the Internet for example, or a combination thereof. Yet other embodiments include checking a patient in manually. According to such other embodiments the receptionist or other technician can enter information related to the patient into the computer terminal 14 using any suitable input peripherals such as a mouse, keyboard, touch-screen interface or a combination thereof. By checking in, the patient's availability for medical treatment at the medical office can be entered into the computer system 10.

The computer system 10 can optionally also include a database server 20, such as a SQL Server connected to a communication network 18 to provide network-accessible storage of at least one of medical database information; and content, such as patient location information for example, to be distributed to the computer terminal 14 or any other network-connected computer resource. Another computer terminal 22 also including a display device 24 is operatively connected to the communication network 18 and disposed at a location within the medical office where technicians can gain access to the display device 24 to determine the location of a waiting patient within the medical office. The location within the medical office at which the computer terminal 22 is located is different than the location of the computer terminal 14, and optionally within a different room or at a different portion of the medical office. For example, the computer terminal 22 can optionally be disposed at a staging area for physicians, nurses or other technicians where they prepare to enter an examination room occupied by a patient. Alternate embodiments include a stand-alone, dedicated display terminal 26 for displaying the GUI described herein, a tablet PC 28 wirelessly connected to the communication network 18, or a combination thereof instead of, or in addition to the computer terminal 22. Yet other embodiments can include any computerized terminal including a display component operable to display a GUI such as that generated according to the method described herein. Each of the computer terminal 22, the display terminal 26, and the tablet PC 28 includes a display component operable to generate the GUI indicating a patient's location using locally-stored content, content served over the mutation network 18 from a remotely-located computer terminal such as a server, or a combination thereof.

Although the examples provided below include a server operatively connected to the communications network 18 to serve content to remotely-located terminals such as computer terminals 14 and 22, the content stored and served by the server can optionally be stored on any network-connected computer device as a shared network resource. For example, the information for tracking a patient's location described herein can be retrieved over the communication network 18 from the shared computer device instead of a network-connected server. According to such embodiments, the information can be retrieved from the shared network resource instead of served by a server to operate in a substantially similar manner as the embodiments described wherein the information is served from a server.

According to an illustrative embodiment the virtual representation of patient location can be presented via the GUI according to the execution of computer-executable logic read from a computer-readable medium. Examples of the computer-readable medium suitable for storing the computer-executable instructions can include a magnetic or solid-state hard disk drive provided to one or more of the computer terminals 14, 22, the database server 20, or other network accessible terminal; a compact disc; a digital versatile disc; EEPROM such as a USB flash drive; and the like.

The patient information stored by the database server 20 or other terminal including a computer-readable medium can optionally store medical database information relating to patients receiving medical care at the medical office. For example, the database server 20 can store a virtual file associated with each patient. The virtual file is said to be virtual in that it exists in an electronic format stored on a computer-readable medium, and is capable of being transmitted in whole or in part over the communication network 18 and at least partially displayed by a display device provided to a computer terminal connected to the communication network 18. The virtual files can optionally include information comparable to that conventionally stored on paper within physical files traditionally stored by the medical office. For instance, each virtual file can include the name of the patient and other personal information of that patient, along with a photograph of the patient associated with that virtual file, medical history information of that patient, and any other information about the patient. The medical database information can optionally include content stored by an existing IT software solution in use with the computer system 10 by the medical office such as that offered under the trade name Centricity by GE Healthcare.

The computer-executable instructions executed to generate the GUI described herein can optionally be installed as a standalone product capable of utilizing information managed by an existing IT solution already installed and operable on the computer system 10 to maintain medical records. Thus, the computer-executable instructions executed in performance of the method described herein minimize interference with existing medical record management systems. The method and system described herein can also optionally retrieve any portion of the medical database information utilized by the existing medical records solution and display such information in monitoring the location of patients as described below.

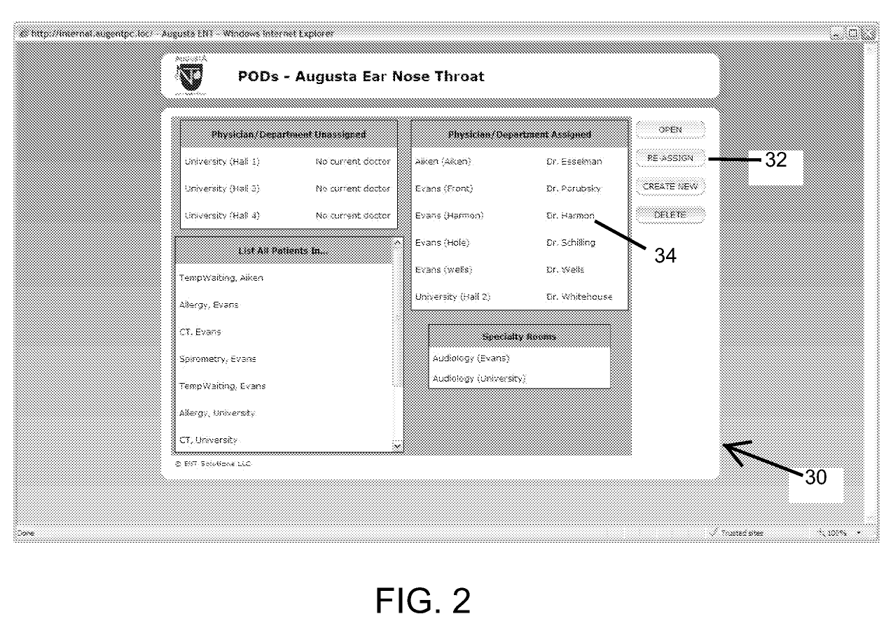

In addition to the medical database information, administrative information regarding the medical office, technicians who interact with patients at the medical office, or a combination thereof is also stored in a computer-readable medium such as the hard disk drive or other computer-readable medium provided to the database server 20. As a specific example, the name of a physician, nurse and/or medical assistant available to see the patient during the administration of medical care can be included in the administrative information. Other embodiments of the administrative information can include the examination room, wing and/or floor of the medical office and/or building where the patient is to be located for receiving medical care, any other geographic information about the medical office and/or its personnel, or a combination thereof can be stored by the database server 20 where it can be accessed and retrieved over the communication network 18. The computer system 10 operating under the control of computer-executable instructions can access the medical database information and the administrative information to generate the virtual representations described with reference to FIGS. 2-5.

FIG. 2 illustrates a GUI administrative window 30 included in the virtual representation generated as described herein. As shown, the administrative window 30 is displayed on the display device 12 of the computer terminal 14 in FIG. 1 within a web-browser software application operating on the computer terminal 14 such as Microsoft Internet Explorer. The administrative window 30 is populated in FIG. 2 by at least a portion of the administrative information retrieved from the database server 20 over the communication network 18. The administrative window 30 allows technicians to add, subtract, view, or otherwise access portions of the administrative information through the use of soft keys 32 also displayed within the administrative window 30. Any changes to the administrative information made via the administrative window 30 can be stored within the database server 20 to update the information already stored therein.

From the GUI shown in FIG. 2 a technician can specify a room, physician, office, or any other type of administrative information is to be the basis for the creation of a GUI for tracking the location of patients associated with the selected administrative information. For example, a technician can select physician entry 34 “Dr. Harmon” from the appropriate menu to generate a GUI for tracking the location of patients arriving at the medical office to be treated by Dr. Harmon.

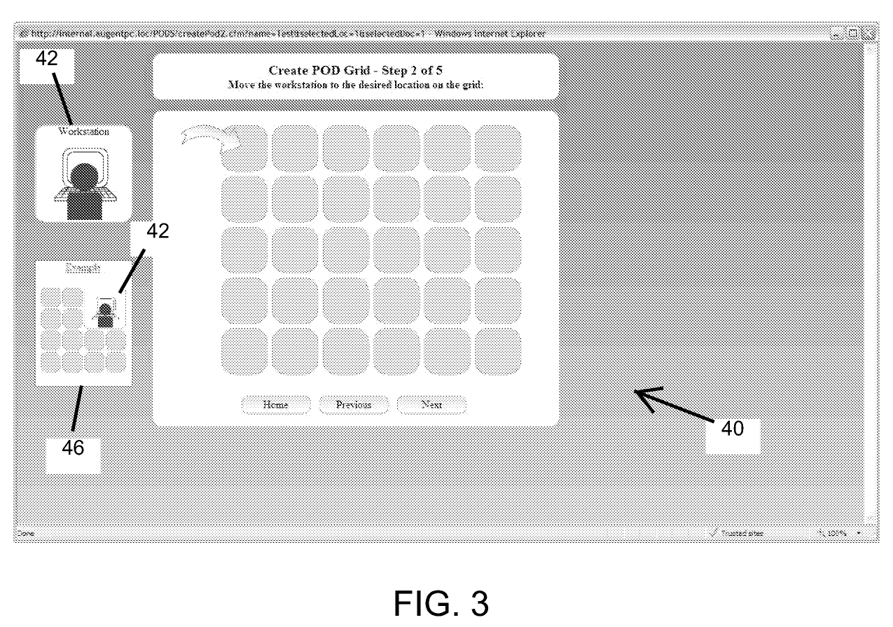

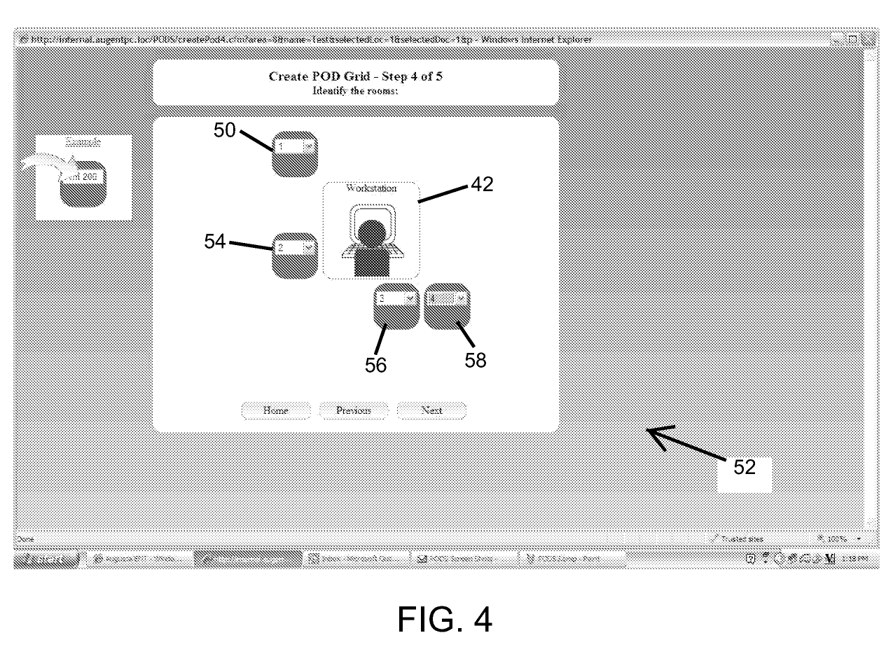

Once the physician or other basis for identifying patients to be tracked has been selected, the method progresses to the layout window 40 shown in FIG. 3. From the layout window 40 the technician is to establish a reference point within the medical office for arranging examination rooms. According to an embodiment, this can be accomplished by dragging a workstation icon 42 to the appropriate location within the layout window 40. The workstation icon 42 can serve to represent any reference point within the medical office. For instance, the workstation icon 42 can optionally represent the location of the workstation in the medical office used primarily by Dr. Harmon. According to alternate embodiments, the workstation icon 42 within the layout window 40 can optionally represent a location of a front desk provided as part of the reception area of the medical office. According to alternate embodiments, the workstation icon 42 does not necessarily represent an actual reference point within the medical office but merely a reference point within the layout window 40 from which the technician, such as the physician treating the patients can readily identify the location of the patient to be treated by looking at the relative positioning of the workstation icon 42 and the various room icons 50, 54, 56, 58 as shown in FIG. 4. An illustrative example of the workstation icon 42 arranged as desired within the layout window 40 is shown in the inset image 46 of FIG. 3.

Once the workstation icon 42 is properly positioned within the layout window 40, the technician can proceed to identify the examination rooms in which patients are to be examined by Dr. Harmon as shown in FIG. 4. According to the illustrative embodiment shown in FIG. 4, the first examination room is represented by room icon 50 within the room identification window 52 of the virtual representation. Likewise, a second examination room is designated by room icon 54, the third examination room is designated by room icon 56 and a fourth examination room in which Dr. Harmon will examine a patient is designated by room icon 58. Again, the arrangement of the room icons 50, 54, 56, 58 relative to the workstation icon 42 in the room identification window 52 can optionally represent a physical layout of the examination rooms 1-4 relative to the physical location of Dr. Harmon's office within the actual medical office.

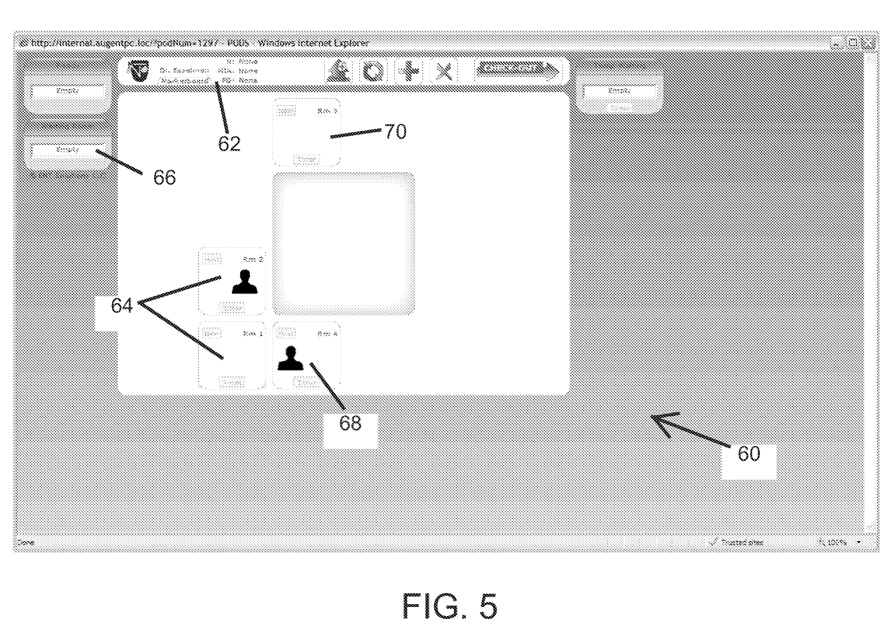

FIG. 5 shows an illustrative embodiment of a monitoring window 60 included in the virtual representation specific to Dr. Esselman as indicated by the information bar 62 adjacent an upper portion of the monitoring window 60. Once arrangement of the room icons 64 relative to the workstation icon 42 in FIG. 5 has been accomplished as described above with reference to FIG. 4, medical database information can optionally be retrieved over the communication network 18 from the database server 20 to populate the monitoring window 60. For instance, the computer system 10 can optionally automatically populate the waiting room field 66, without intervention by a technician according to an embodiment, in response to a patient being checked in at the reception area of the medical office, such as by scanning an ID card carried by the patient via the scanner 16 (FIG. 1).

Any information identifying the patient can be populated within the waiting room field 66 to represent the location of the patient within a waiting room of the medical office. According to an illustrative embodiment, the patient's name along with a photograph of the patient retrieved over the communication network 18 from the database server 20 can be populated within the waiting room field 66. When the patient is escorted from the waiting room and into an examination room, such as the examination room represented by examination room icon 68 for the example shown in FIG. 5, a receptionist or other technician aware of the patient's movement can move the representation of the patient (i.e., the patient's name and photograph) from the waiting room field 66 to the room icon 68.

Moving the representation of the patient can optionally be accomplished by performing a so-called “drag and drop” operation. The technician can select the patient by placing a cursor displayed by the computer terminal 14 on the representation within the waiting room field 66. While pressing and holding a mouse button the technician can reposition the representation of the patient onto room icon 68. Room icon 68, in response, undergoes a change in appearance such as showing a photograph of the patient within the room icon 68, listing the patient's name within the room icon 68, listing an appointment time at which the patient is scheduled to receive medical attention within the room icon 68, providing a brief description of the patient's reason for visiting a medical office within the room icon 68, or a combination thereof. Any information displayed within the room icon 68 can optionally be hidden from view until the room icons 68 is selected by the technician such as by double-clicking on the room icons 68 using a mouse operatively connected to the computer terminal being used to display the monitoring window 60. In response to selection of the room icons 68 the hidden information can be presented within the virtual presentation to notify the technician of the reason for the patient's visit or other information relating to that patient.

In the event that the patient in the examination room represented by room icon 68 in FIG. 5 is moved to a different examination room, the monitoring window 60 can be updated to reflect this move. For example, the technician who escorted the patient from one examination room to the examination room represented by room icon 70 can approach the computer terminal 22 in FIG. 1 and perform another drag and drop operation to move the representation of the patient from room icon 68 to room icon 70. According to an alternate embodiment, receptionist can optionally perform a similar drag-and-drop operation via the computer terminal 14 to reflect moving the patient to a different examination room. Changes to the GUI shown in FIG. 5 can optionally be made in any computer terminal with the required authorization to access the data displayed. Thus, updates to patient locations can be graphically displayed in substantially real-time to minimize confusion regarding the location of patients within the medical office.

When the physician or other technician is ready to examine the patient, the exact location of the patient can be determined by simply observing the monitoring window 60 shown in FIG. 5 via the computer terminals 14, 22, a standalone display terminal 26, the portable display device such as the tablet PC 28 carried by the technician, any other display device, or a combination thereof. Following examination of the patient, a physician that examined the patient or any other technician can access any of the computer terminals 14, 22, display terminal 26, the tablet PC 28, or any other networked computer terminal to remove the patient from the monitoring window 60 indicating that the patient has left the medical office, or is at least finished receiving medical attention. All changes made to the monitoring window 60 can optionally be updated at the database server 20 and propagated throughout the computer system 10 to provide a substantially real-time indication of a patient's location within the medical office.

The illustrative embodiments discussed above are mostly implemented by computer executable instructions read from a computer-readable medium. Further, the various virtual presentations described above can be implemented using an interactive website via a user computer connected to the Internet. In this manner, a technician can monitor and track a patient's location outside the medical office, such as in another building on a medical campus for example.

Throughout this disclosure, the term “virtual” is used, for example, to describe the user viewable/hearable material presented to the user on a display device from data and/or computer programs and commands generated and/or provided by the computer system 10. According to alternate embodiments the data and/or programs, or at least a portion thereof, can be stored locally by a computer terminal displaying the virtual presentation to the technician.

Still further provided is a computer readable medium for storing computer readable program code for performing the method disclosed herein by utilizing a computer system, as also disclosed herein.

As used herein, the terms “component” and “system” are intended to refer to a computer-related entity, either hardware, a combination of hardware and software, software, or software in execution. For example, a component may be, but is not limited to being, a process running on a processor, a processor, an object, an executable, a thread of execution, a program, and a computer. By way of illustration, both an application running on a server and the server can be a component. One or more components may reside within a process and/or thread of execution and a component may be localized on one computer and/or distributed between two or more computers.

The invention has been described hereinabove using specific examples; however, it will be understood by those skilled in the art that various alternatives may be used and equivalents may be substituted for elements or steps described herein, without deviating from the scope of the invention. Modifications may be provided to adapt the invention to a particular situation or to particular needs without departing from the scope of the invention. It is intended that the invention not be limited to the particular implementation described herein.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method of tracking a patient at a medical facility, the method comprising:
receiving, using a computer, a first location to be occupied by the patient within the medical facility;
storing the first location in a non-transient computer-readable memory in communication with a remotely-located computer terminal over a communication network;
transmitting content indicating that the patient is to occupy the first location over the communication network to the remotely-located terminal to be displayed by the remotely-located terminal, wherein the remotely-located terminal displays a graphical representation that the patient is to occupy the first location in response to receiving the content;
receiving a second location to be occupied by the patient within the medical facility, wherein the second location is different than the first location;
in response to receiving the second location, storing the second location occupied by the patient in the computer-readable memory; and
transmitting content indicating that the patient is to occupy the second location over the communication network to the remotely-located terminal for updating the graphical representation displayed by the remotely-located terminal to reflect a change of the location of the patient from the first location to the second location.

2. The method according to claim 1, wherein the computer-readable memory storing the first and second locations is remotely-located from the computer and is operatively connected to the computer over the communication network.

3. The method according to claim 1 further comprising establishing an arrangement of a plurality of examination locations within the medical facility where the patient can be moved to receive medical attention, wherein each of the plurality of examination locations is represented by a room icon, and the room icons are arranged to generally correspond to a physical layout of the medical facility.

4. The method according to claim 3, wherein an occupied room icon representing an occupied one of the plurality of examination locations to be occupied by the patient has an appearance in the graphical representation displayed by the remotely-located terminal that is different than an unoccupied room icon representing another of the plurality of examination locations that is not occupied by the patient.

5. The method according to claim 1, wherein the content transmitted over the communication network comprises an identification of an examination location to be occupied by the patient and an identification of the patient.

6. The method according to claim 5, wherein the identification of the patient comprises at least one of a photograph of the patient, a graphical depiction of the patient that is not a photograph of the patient, a patient name, and a patient ID number.

7. The method according to claim 1, wherein receiving the second location comprises:
including a drag-and-drop interface in the graphical representation to be manipulated by a user controlling a peripheral input device in communication with at least one of the computer and the remotely-located terminal to indicate the change of location of the patient, wherein the drag-and-drop interface comprises an occupancy graphic corresponding to the patient that can be selected by the user via the peripheral input device and relocated to a portion of the graphical representation representing the second location; and
in response to relocation of the occupancy graphic, receiving content to indicate the change of location of the patient from the first location to the second location.

8. The method according to claim 1, wherein the first location comprises a waiting room at the medical facility where the patient awaits medical attention and the second location comprises an examination room at the medical facility where the patient is to receive medical attention.

9. The method according to claim 1 further comprising:
transmitting over the communication network content indicating a length of time that the patient has waited to receive medical attention since the patient registered arriving at the medical facility;
transmitting an alert to a worker at the medical facility that the patient has arrived; and
including in the graphic displayed by the remotely-located terminal the length of time that the patient has waited to receive medical attention.

10. A method of tracking a patient at a medical facility, the method comprising:
using a computer comprising an input peripheral manipulated by a user, receiving a first location to be occupied by the patient within the medical facility, the first location being identified by the user via the input peripheral;
displaying the first location on a display device operatively connected to the computer, the first location being identified by a graphical marker representing the first location where the patient can be found and an identification of the patient;
in response to a user interaction with the input peripheral, receiving a second location of the patient indicating a change of location of the patient from the first location to the second location, wherein the second location is different than the first location; and
in response to receiving the second location, moving the graphical marker on the display device operatively connected to the computer to identify the second location of the patient.

11. The method according to claim 10, wherein the user interaction with the imputer peripheral comprises selecting and dragging the graphical marker displayed by the display device from the first location to the second location.

12. The method according to claim 10 further comprising displaying a plurality of room icons, each of the room icons representing a different one of a plurality of examination rooms at the medical facility, wherein the graphical marker includes an occupied room icon representing one of the plurality of different examination rooms where the patient is to be located and has an appearance that is different than an appearance of a room icon representing another one of the examination rooms where the patient is not to be located.

13. The method according to claim 12, wherein the graphical marker comprises at least one of: a picture of the patient, a name of the patient, a patient ID number of the patient, and an indication of a length of time that the patient has been waiting for medical attention.

14. A system for tracking a location of a patient comprising:
a computer operatively connected to a communication network;
a remotely-located computer operatively connected to the communication network, wherein the computer and the remotely-located computer each comprise a display device and are disposed at different locations within a medical facility; and
a non-transient, computer-readable memory that is accessible by at least one of the computer terminal and the remotely-located computer terminal over the communication network, wherein the computer-readable memory is operable to store the location of the patient within the medical facility, and wherein at least one of the computer and the remotely-located computer comprise a memory storing computer-executable instructions to be executed for performing a method comprising:
receiving content over the communication network indicative of the location of the patient within the medical facility;
in response to receiving the content, presenting a graphical representation of a plurality of different examination locations at the medical facility, each of the plurality of different examination locations being represented by a room icon, wherein an occupied room icon representing one of the plurality of examination locations to be occupied by the patient has an appearance that is visually distinguishable from an unoccupied room icon displayed to represent another of the plurality of examination locations not occupied by the patient;
receiving, via an input peripheral, an instruction that the location of the patient is to be changed from a first one of the examination locations to a second one of the examination locations; and
transmitting information indicative of the change of the location over the communication network to automatically update the graphical representation of the plurality of different examination locations displayed by the remotely-located computer terminal without user interaction with the remotely-located computer terminal.

15. The system according to claim 14, wherein the computer and the remotely-located computer are disposed at different locations at the medical facility.

16. The system according to claim 14, wherein receiving the instruction that the location of the patient is to be changed comprises:
rendering the occupied room icon as a drag-and-drop computer-generated graphic that can be moved to a plurality of different locations by a user via the input peripheral; and
changing an appearance of a previously unoccupied room icon to which the user has dragged the occupied room icon to reflect the change of the location of the patient.

17. The system according to claim 14, wherein the computer-executable instructions, when executed, cause the appearance of the occupied room to comprise an identification of the examination location to be occupied by the patient and an identification of the patient.

18. The method according to claim 17, wherein the identification of the patient comprises at least one of a photograph of the patient, a patient name, and a patient ID number.
